(19) United States
(12) Patent Application Publication
(10) Pub. No.: US 2024/0417680 A1
(43) Pub. Date: Dec. 19, 2024

(54) NANOTUBE COMPOSITIONS AND METHODS FOR FACILIATING STEM CELL GROWTH

(71) Applicant: Molecular Rebar Design, LLC, Houston, TX (US)

(72) Inventors: Kevin R. Castillo, Austin, TX (US); Aaron Z. Foote, Austin, TX (US); Conner J. Quinian, Lakeway, TX (US); Clive P. Bosnyak, Dripping Springs, TX (US); Kurt W. Swogger, Dripping Springs, TX (US)

(21) Appl. No.: 18/745,344

(22) Filed: Jun. 17, 2024

Related U.S. Application Data
(60) Provisional application No. 63/521,451, filed on Jun. 16, 2023.

(51) Int. Cl. C12N 5/00; C12N 5/0735

(52) U.S. Cl. CPC C12N 5/0068; C12N 5/0606; C12N 2533/30; C12N 2533/90

(57) ABSTRACT

The application pertains to compositions and methods useful for growing living cells such as stem cells. The compositions employ a mixture of an extracellular matrix and discrete carbon nanotubes. The extracellular matrix may also comprise components selected from the class of proteins, proteoglycans, polysaccharides, lipids, peptides, messenger molecules, signaling molecules, or any mixture thereof. The discrete carbon nanotubes are usually less than about 1% by weight of the dry weight of the total composition.

### CROSS-REFERENCE TO RELATED APPLICATIONS

The present application claims priority to U.S. provisional application Ser. No. 63/521,451 filed Jun. 16, 2023 which application is incorporated herein by reference.

### FIELD OF DISCLOSURE

The present disclosure relates generally to compositions and methods employing extracellular matrices, ECM, with less than about 1% by weight discrete carbon nanotubes to facilitate increases in living cell proliferation and/or living cell lifespan.

### BACKGROUND

There is much need for increases in the proliferation of living cells, such as, but not limited to stem cells, and/or living cell lifespan. The potential of multipotent stem cells as a treatment for a wide variety of diseases and injuries has been established in recent years. When transplanted into a tissue or organ system, multipotent stem cells are able to differentiate into the local cell type, divide, and ultimately regenerate the diseased or wounded tissue.

One problem facing both the development and implementation of stem cells as a treatment is their relatively slow proliferation rate. To serve as a successful treatment, stem cells must be grown in large numbers to regenerate tissue and integrate into existing organ systems. The amount of time required for primary stem cells to proliferate to a usable level is a major bottle neck both in stem cell treatments and research.

The extracellular matrix (ECM) that is used to grow living cells such as, but not limited to stem cells, consists of such materials, but not limited to, fibrous proteins, macromolecules, sugars, lipids and cell signaling molecules, and plays a major role in stem cell health, differentiation, and proliferation. The biochemical and biophysical properties of the ECM are carefully regulated by cells and tissues to produce favorable outcomes in cell behavior and growth. The physical and chemical properties of the extracellular matrix such as, but not limited to, mechanical strength rigidity and electrical conductivity, as well as the concentration of a wide range of growth factors and signaling molecules can all impact cell to cell and cell to matrix interactions.

The extracellular matrix (ECM) is the non-cellular component present within all living tissues and organs and provides not only essential physical scaffolding for the cellular constituents, but also initiates crucial biochemical and biomechanical cues that are required for tissue morphogenesis, differentiation and homeostasis. The importance of the ECM is illustrated by the wide range of syndromes, which can be anything from minor to severe, that arise from genetic abnormalities in ECM proteins. Although, fundamentally, the ECM is composed of water, proteins and polysaccharides, each tissue has an ECM with a unique composition and topology that is generated during tissue development through a dynamic and reciprocal, biochemical and biophysical dialogue between the various cellular components (e.g., epithelial, fibroblast, adipocyte, endothelial elements) and the evolving cellular and protein microenvironment. Indeed, the physical, topological, and biochemical composition of the ECM is not only tissue-specific but is also markedly heterogeneous. Cell adhesion to the ECM is mediated by ECM receptors, such as integrins, discoidin domain receptors and syndecans. Adhesion mediates cytoskeletal coupling to the ECM and is involved in cell migration through the ECM. Moreover, the ECM is a highly dynamic structure that is constantly being remodeled, either enzymatically or non-enzymatically, and its molecular components are subjected to a myriad of post-translational modifications. Through these physical and biochemical characteristics, the ECM generates the biochemical and mechanical properties of each organ, such as its tensile and compressive strength and elasticity, and mediates protection by a buffering action that maintains extracellular homeostasis and water retention. In addition, the ECM directs essential morphological organization and physiological function by binding growth factors (GFs) and interacting with cell-surface receptors to elicit signal transduction and regulate gene transcription. The biochemical and biomechanical, protective and organizational properties of the ECM in a given tissue can vary tremendously from one tissue to another (e.g., lungs versus skin versus bone) and even within one tissue, as well as from one physiological state to another (normal versus cancerous).

Modifications to the ECM can have beneficial effects on embedded stem cells. However, it is critical that these modifications are carefully adjusted with precision given the sensitivity of stem cells to the properties of their local environment. Additionally, modifications to the ECM would best be made by a versatile additive or method capable of altering several properties simultaneously without impacting toxicity.

Variations in the ECM can contribute to the health of embedded cells, as they can support normal cell behavior or induce cancer, and other harmful conditions. External modifications of the ECM can induce positive effects, such as eliminating a disease state of the surrounding cells and tissues or increasing a beneficial parameter of the given microenvironment.

Discrete carbon nanotubes of this invention can be integrated into the ECM to improve the rate of cell proliferation. Conversely, commercially available carbon nanotubes can present several problems in biological applications. Products such as CNano Flowtube carbon nanotubes are typically produced in bundles 2-500 μm in size wherein the carbon nanotubes are highly entangled. Single wall carbon nanotubes are energetically favored to align and so form ropes, containing thousands of single wall carbon nanotubes. These ropes can entangle together to form bundles. Lastly, metal catalysts that are necessary for carbon nanotube synthesis can be encased within bundles, introducing potentially toxic elements for cells and tissues.

U.S. Pat. No. 10,414,655 by Bosnyak et al. discloses stem cell, bone and nerve scaffolding comprising discrete carbon nanotubes in the weight fraction of greater than 1% by weight of the scaffold. The discrete carbon nanotubes have targeted, or selective oxidation levels and/or content on the interior and exterior of the tube walls as described in U.S. Pat. No. 10,414,656 which is incorporated herein by reference. The invention disclosed herein differs in one aspect from U.S. Pat. No. 10,414,655 in that the amount of discrete carbon nanotubes used to augment the ECM can be less than about 1% by weight, preferably less than about 0.1% by weight, of an extracellular matrix or scaffold and yet unexpectedly exhibit enhanced cell growth. At less than about 1% by weight of the extracellular matrix the discrete carbon nanotubes could be dispersed so that the individual carbon nanotubes are not in contact with another discrete carbon nanotube to form a supported structure or scaffold.

Provided in the present embodiments are new compositions and methods that provide substantial increases in cell growth using a relatively small amount of nanotubes.

### SUMMARY OF THE INVENTION

A composition is disclosed herein comprising a mixture of an extracellular matrix and discrete carbon nanotubes wherein the extracellular matrix comprises components selected from the class of proteins, proteoglycans polysaccharides, lipids, peptides, messenger molecules, signaling molecules, or any mixture thereof, and wherein the discrete carbon nanotubes are less than about 1% by weight of the dry weight of the total composition. The composition can further comprise living cells. The living cells could be stem cells or primary stem cells.

At least a portion of the extracellular matrix, or at least a portion of the discrete carbon nanotubes, or both, can be at least partially oriented which could promote directional growth of living cells.

In the composition at least a portion of the discrete carbon nanotubes can be physically adhered or chemically bonded to at least one component of the composition.

The composition can be subjected to a static current, an oscillating current, a static voltage, an oscillating voltage, or any combination thereof to stimulate living cell growth.

The composition comprising a mixture of an extracellular matrix and discrete carbon nanotubes may contain discrete carbon nanotubes that are substantially individualized from other discrete carbon nanotubes in the extracellular matrix.

The composition comprising a mixture of an extracellular matrix and discrete carbon nanotubes the composition further comprises a dispersing agent wherein the dispersing agent substantially prevents the discrete carbon nanotubes from agglomerating. The dispersing agent may be physically or chemically attached to the discrete carbon nanotubes and selected form the class of anionic, cationic, non-ionic and zwitterionic molecules.

The composition may further comprise at least a portion of the discrete carbon nanotubes that further comprise a molecule distinct in structure from the components of the extracellular matrix wherein the molecule is at least partially bound to the nanotube surface and is selected from the class of molecules that function as an antibacterial, a growth factor, a growth stimulant, a nutrient, or combinations thereof.

Additional fillers may be added to the composition such as, but not limited to graphene, graphene oxide, carbon nanofibers, cellulose nanofibers, hydroxy apatite, silk, and mixtures thereof. These additional fillers may serve to change the rigidity or porosity of an ECM. The discrete carbon nanotubes may associate with these additional fillers.

The composition may be in the form of a closed cell foam, an open cell foam, a film, a strand, particles, or mixtures thereof. Open cell foam structures are preferred to allow nutrients and air to reach living cells.

The composition may further comprise water.

Methods for growing living cells together with the composition of this invention are disclosed.

The composition has utility for, but not limited to, injury, burns, wound care, sensors, bone, skin, muscle, nerve, blood, and organ treatment for accelerated repair of damage compared to a similar composition of the extracellular matrix without discrete carbon nanotubes. The composition may be of any suitable form for application and can be shaped, injected, implanted or fitted.

The composition may include discrete carbon nanotubes (dCNTs) that are single walled (dSWCNTs), double walled (dDWCNTs) or multiwalled (dMWCNTs) which may or may not be oxidized or functionalized.

### BRIEF DESCRIPTION OF THE FIGURES

In order to facilitate a fuller understanding of the present invention, reference is now made to the attached figures. The figures should not be construed as limiting the present invention but are intended only to illustrate different aspects and embodiments of the invention.

FIG. 1 illustrates the improvement in cell proliferation stem cells experience when incubated in a 3D ECM-like matrix containing dMWCNTs. One sample of mesenchymal stem cells were grown in Vitrogel while another sample was grown in Vitrogel that had been incubated with dMWCNTs at a concentration of 0.001 mg/ml. Each condition was plated at cell concentration of 5.0×10\hat{ }5 per well in a 24-well plate and then incubated for 10 days in a 37° C., 5% CO2 incubator. Thereafter the viable cells were extracted and counted using a bio-active colorimetric assay (WST-8). This assay uses a water-soluble tetrazolium salt to quantify the number of live cells by producing an orange formazan dye upon bio-reduction in the presence of an electron carrier, a marker for metabolic activity.

FIG. 2 illustrates how stem cell health and rate of proliferation are increased when incubated in a 3D, dMWCNT infused ECM-like scaffold. In this example, the dMWCNTs are dispersed in DSPE-PEG(NH₂) in a CNT:PEG ratio of 1:0.55. For comparison, stem cells incubated in a 3D ECM-like scaffold without dMWCNTs and with only DSPE-PEG(NH₂) are shown. Scale bar is 100 μm, and all conditions were incubated for 9 days before imaging.

FIG. 3 shows brightfield microscopy images of each concentration of dMWCNTs in Vitrogel.

FIG. 4 shows single walled carbon nanotubes noncovalently dispersed in DSPE-PEG(NH₂).

### DETAILED DESCRIPTION

Exemplary embodiments of the invention will now be described in order to illustrate various features of the invention. The embodiments described herein are not intended to be limiting as to the scope of the invention, but rather are intended to provide examples of the components, use, and operation of the invention.

Furthermore, the described features, advantages, and characteristics of the embodiments may be combined in any suitable manner. One skilled in the relevant art will recognize that the embodiments may be practiced without one or more of the specific features or advantages of an embodiment. In other instances, additional features and advantages may be recognized in certain embodiments that may not be present in all embodiments.

The present embodiments relate generally to a mixture of an extracellular matrix and discrete carbon nanotubes wherein the extracellular matrix comprises components selected from proteins, proteoglycans polysaccharides, lipids, peptides, messenger molecules, signaling molecules, or any mixture thereof, and wherein the discrete carbon nanotubes are less than about 1% of the dry weight of the total composition, preferably less than about 0.05%, more preferably less than about 0.01% and most preferably less than about 0.001% of the dry weight of the total composition.

Discrete carbon nanotubes, dCNT, serving an additive component of an extracellular matrix used in this invention can consist of single wall, double wall or multiwall graphene shells, or mixtures thereof.

“Discrete Carbon Nanotubes (dCNT)” refers to carbon nanotubes that have a well-defined and measurable length, capable of being unbundled or untangled so as to be substantially separated from one another along the length of the carbon nanotube. The discrete carbon nanotubes can be individually dispersed in a given medium by selection of the thermodynamic interaction of the tube surface, the medium and the tube concentration in the medium.

During the process of making discrete or exfoliated carbon nanotubes (which can be single, double and multiwall configurations or mixtures thereof), from bundles or entangled masses of carbon nanotubes, the nanotubes can be cut into segments with at least one open end and residual catalyst particles that are exterior and/or interior to the carbon nanotubes can be removed, for example with acids such as nitric acid. The cutting of the carbon nanotube can be performed by high energy mechanical means and/or chemical means. Discrete carbon nanotubes with smaller aspect ratios are easier to exfoliate than those with larger aspect ratios. Exfoliation here meaning that the discrete carbon nanotubes can be separated as individual carbon nanotubes. Proper selection of the carbon nanotube feed stock related to catalyst particle type and distribution in the carbon nanotubes allows more control over the resulting individual tube lengths and overall tube length distribution. A preferred selection is where the internal catalyst sites are evenly spaced and where the catalyst is most efficient. A further preferred selection is where there are Stones-Wales defects present along the wall or walls of the carbon nanotube. Individual discrete carbon nanotubes can have an aspect ratio of from about 5 to about 10,000, preferably about 25 to about 2000, more preferably about 25 to about 500 and most preferably about 50 to about 200. The selection of discrete carbon nanotubes for the extracellular matrix can be evaluated using electron microscopy and determination of the discrete or exfoliated carbon nanotube lengths and length distribution.

Discrete oxidized carbon nanotubes are obtained from as-made bundled or entangled carbon nanotubes by methods involving oxidation, such as, but not limited to, using concentrated nitric acid, a combination of concentrated sulfuric and nitric acids, or peroxides. The bundled or entangled carbon nanotubes can be made from any known means such as, for example, chemical vapor deposition, laser ablation, and high-pressure carbon monoxide synthesis. It is preferred that the carbon nanotubes are made via catalysts that are non-toxic, for example iron, rather than for example cobalt. The bundled carbon nanotubes can be present in a variety of forms including, for example, soot, powder, fibers, and bucky paper. Furthermore, the bundled carbon nanotubes may be of any length, diameter, or chirality. Carbon nanotubes may be metallic, semi-metallic, semi-conducting, or nonmetallic based on their chirality and number of walls. The discrete carbon nanotubes may include, for example, single-wall, double-wall carbon nanotubes, or multi-wall carbon nanotubes or combinations thereof. One of ordinary skill in the art will recognize that many of the specific aspects of this invention illustrated utilizing a particular type of carbon nanotube may be practiced equivalently within the spirit and scope of the disclosure utilizing other types of carbon nanotubes. Other types of carbon nanotubes, for example, include boron.

A preferred selection of carbon nanotubes of this invention is the incorporation of a portion of structures called Stone-Wales defects which are the rearrangement of the six-membered rings of graphene into heptagon-pentagon pairs that fit within the hexagonal lattice of fused benzene rings constituting a wall of the carbon nanotubes. These Stone-Wales defects are useful to create sites of higher bond-strain energy for more facile reaction such as oxidation of the graphene or carbon nanotube wall. These defects and other types of fused ring structures may also facilitate bending or curling along the length of the carbon nanotubes which is advantageous for maintaining fluidity of mixtures of discrete carbon nanotubes.

Stone-Wales defects are thought to be more prevalent at the end caps that allow higher degrees of curvature of the walls of carbon nanotubes. During oxidation the ends of the carbon nanotubes can be opened and can also result in higher degrees of oxidation than along the walls. The higher degree of oxidation and hence higher polarity or hydrogen bonding at the ends of the tubes are considered useful to help decrease the average contour length to end to end length ratio when the tubes are present in water and other polar media.

According to a preferred embodiment, the discrete carbon nanotubes are further functionalized. The functional groups linked to dCNT include, but not limited to, hydroxyl, thiol, amide, amine and carboxyl groups. The functionalized dCNT allows for the decrease in aggregation between dCNT molecules in a medium. The functionalization allows for interaction with inorganic metals, inorganic salts, organic molecules such as, but not limited to comprising ether, or imine, or amide, or ester groups, and biological species such as, but not limited to DNA, RNA, peptides, proteins, and enzymes. The discrete carbon nanotubes may be associated or bound to polymers such as poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), and poly(N-acryloylmorpholine) (PACM). Particularly useful polymer molecules for functionalizing the discrete carbon nanotubes are the modified polyethylene oxide copolymers such as, but not limited to 1,2-distearoyl-sn-glycero-3-phosphoethanolamine-N-amino (polyethylene glycol), DSPE-PEG-NH₂.

Other functionalized carbon nanotubes of the present disclosure generally refer to the modification of any of the carbon nanotube types described hereinabove. Such modifications can involve the nanotube ends, sidewalls, or both. Modifications may include, but are not limited to covalent bonding, ionic bonding, chemisorption, intercalation, surfactant interactions, polymer wrapping, cutting, solvation, and combinations thereof.

At least a portion of discrete nanotubes identified preferably has a ratio of number average value of tube contour length (TCL)):tube end to end length (TEE) of from about 1.1 to about 3, preferably from about 1.1 to about 2.8, more preferably from about 1.1 to about 2.4, most preferably from about 1.1 to about 2 and especially form about 1.2 to about 2. The values of TCL and TEE can be measured by scanning electron microscopy.

The ratio of the TCL to TEE can be advantageously controlled by the degree of thermodynamic interaction between the tube surfaces and the medium. Surfactants can be usefully employed also to modify the thermodynamic interactions between the tubes and the medium of choice. Alternate means to influence the ratio of discrete carbon nanotube contour length to end to end ratio include the use of inorganic or ionic salts such as sodium chloride and organic containing functional groups with oxygen or nitrogen moieties.

Yet another method for controlling the TCL to TEE ratio is by orientation. The discrete carbon nanotubes can be oriented by such means, although not limited, by extrusion through a circular or slit die, drawing fibers, or through foaming, or blowing films, injection or tentering. The orientation is facilitated by the presence of a polymer in the fluid. An example of this is employing discrete carbon nanotubes of this invention in the presence of polyvinyl alcohol and water such that polyvinyl alcohol-oriented discrete carbon nanotube fibers can be obtained by electrospinning or via orifices in the wall of a spinning centrifuge.

The carbon nanotubes used in the invention described herein need not comprise 100% discrete carbon nanotubes. That is, some carbon nanotube bundles may still exist as entangled, non-discrete tubes. However, the carbon nanotubes used in this ECM invention comprise at least 70% wt. based on the whole carbon nanotube compositions of discrete carbon nanotubes, more preferably greater than 80%, most preferably greater than 95% and especially 99% or more discrete carbon nanotubes.

The composition comprising discrete carbon nanotubes can further comprise polymers selected from a variety of natural, synthetic, and biosynthetic polymers that are biocompatible or biodegradable. A polymer based on a C-C backbone tends to resist degradation, whereas heteroatom-containing polymer backbones confer biodegradability. Biodegradability can, therefore, be engineered into polymers by the judicious addition of chemical linkages such as anhydride, ester, or amide bonds, among others. The usual mechanism for degradation is by hydrolysis or enzymatic cleavage of the labile heteroatom bonds, resulting in a scission of the polymer backbone. Macro organisms can eat and, sometimes, digest polymers, and also initiate a mechanical, chemical, or enzymatic aging. Biodegradable polymers with hydrolysable chemical bonds are researched extensively for biomedical, pharmaceutical, agricultural, and packaging applications. In order to be used in medical devices and controlled-drug-release applications, the biodegradable polymer must be biocompatible and meet other criteria to be qualified as biomaterial-processable, sterilizable, and capable of controlled stability or degradation in response to biological conditions. The chemical nature of the degradation products, rather than of the polymer itself, often critically influences biocompatibility. Poly(esters) based on polylactide (PLA), polyglycolide (PGA), polycaprolactone (PCL), and their copolymers have been extensively employed as biomaterials. Degradation of these materials yields the corresponding hydroxy acids, making them safe for in vivo use. Other bio- and environmentally degradable polymers include polyvinyl alcohols, poly(hydroxyalkanoate) s such as the polyhydroxybutyrate-polyhydroxyvalerate class, additional poly(ester)s, and natural polymers, particularly, modified poly(saccharide)s, e.g., starch, cellulose, and chitosan.

The rigidity of the ECM containing discrete carbon nanotubes can be selected based on the choice of polymer, the ratio of polymer to discrete carbon nanotube and the density of the scaffold. The rigidity of the scaffold can determine the type of cell the stem cells differentiate towards.

The ECM containing discrete carbon nanotubes can further comprise other additives conducive for directing stem cell differentiation such as other inorganic types like graphite, graphene, and silicate structures, polymeric additives for example but not limited to classes of polymers such as polyolefins and natural products such as, but not limited to silk or DNA strands.

Stem cells applied to this invention are not restricted, including any stem cell having inherent characteristics such as non-differentiation, infinite proliferation and differentiative potential to specific cell types. The preferable stem cells used in this invention are classified into two groups: pluripotent stem cells such as embryonic stem cells and embryonic germ cells; and multipotent stem cells. Embryonic stem cells are derived from inner cell mass of blastocyst, and embryonic germ cells are derived from primordial germ cells present in 5-10 week aged gonadal ridge. Multipotent stem cells are found in embryonic tissues, fetus tissues or adult tissues, including adult (somatic) stem cells. Pluripotent stem cells are proliferated in vitro and differentiate to three germ layers (ectoderm, mesoderm and endoderm). It is unlikely that multipotent stem cells have the capability to differentiate to their precursor tissues, and their self-renewal potency is restricted. The source of multipotent stem cells includes any type of tissues, in particular, bone marrow, blood, liver, skin, intestine, spleen, brain, skeletal muscle and dental pulp.

Preferably, stem cells used in this invention are embryonic stem cell, adult stem cell, embryonic germ cell and embryonic carcinoma cell, more preferably, embryonic stem cell and adult stem cell.

The ECM can contain discrete carbon nanotubes that are adhered to the constitutive elements of the matrix so that the dCNTs are not removed upon washing or other forms of transfer of aqueous fluid. The nanotubes can be adhered to the constitutive elements of the ECM at a single point of contact, all along the length of the nanotube, or any amount in between. These adherent forces may include but are not limited to Van der Waals forces, hydrogen bonding, depletion forces, ionic bonding, etc.

The dCNTs may be adhered to the constitutive elements of the ECM in any orientation as long as the dCNTs are not removed from the matrix upon washing. These orientations include but are not limited to wrapped around ECM structures, adhered along one or multiple ECM structures, fixed at one end of the nanotube while the other diffuses freely, etc.

The composition comprising a mixture of ECM and discrete carbon nanotubes may comprise lengths of the discrete carbon nanotubes that can be a unimodal distribution, or a multimodal distribution (such as a bimodal distribution). The multimodal distributions can have evenly distributed ranges of lengths (such as 50% of one length range and about 50% of another length range). The distributions can also be asymmetrical-meaning that a relatively small percent of discrete nanotubes can have a specific length while a greater amount can comprise another length.

The composition comprising a mixture of ECM and discrete carbon nanotubes wherein the diameters of the discrete carbon nanotubes can be a unimodal distribution, or a multimodal distribution (such as a bimodal distribution). The multimodal distributions can have evenly distributed ranges of diameters (such as 50% of one diameter range and about 50% of another diameter range). The distributions can also be asymmetrical-meaning that a relatively small percent of discrete nanotubes can have a specific diameter while a greater amount can comprise another diameter.

The composition comprising a mixture of ECM and discrete carbon nanotubes can contain discrete carbon nanotubes wherein the discrete carbon nanotubes can comprise combinations of functionality. One example, but not limiting, is a portion of discrete carbon nanotubes having 2% by weight of carboxylic acid groups may be admixed with a portion of discrete carbon nanotubes having 2% by weight hydroxyl groups for the purpose of providing association or chemical bonding to different components of an ECM.

The carbon nanotubes can be contacted with surfactants or dispersing agents selected from the class of anionic, cationic, non-ionic or zwitterionic molecules, and combinations thereof. An example of an anionic surfactant is sodium dodecylsulfate. An example of a cationic surfactant is cetyltrimethylbromide. An example of a non-ionic surfactant is Pluronic F127, a block polyethylene oxide-polypropylene oxide. An example of a zwitterionic surfactant is dioctanoyl phosphatydyl choline.

The ECM for transplanting stem cells containing dCNT shows excellent properties in networking between differentiated stem cells and surrounding cells. The dCNT ECM exhibits improved cell adhesiveness to improve cell density and cell-to cell adhesion, and no cytotoxicity. Such features of dCNT contribute to the formation of networking between stem cells transplanted and surrounding tissues, allowing stem cells transplanted to exert their functions and effects. Furthermore, such features prevent stem cells transplanted from being washed away.

In particular, the mixture for transplanting stem cells comprising dCNT can be easily transplanted in a fluid form, for example using a syringe. The stem cells can be surrounded by a dispersion of discrete carbon nanotubes to protect the stem cell during fluid transport.

Since the dCNT containing ECM is well mixed with stem cells and injected into sites of interest, it can decrease adverse effects associated with surgical procedures. The injected dCNT may form structures suitable in the formation of cell-to-cell networks over time. The electric conductivity of dCNT permits it to be delivered to sites of interest via electric induction, thereby making it possible to serve as stem cell compositions at sites with disrupted tissues.

The diseases or disorders treated by the present composition comprise all diseases or disorders that can be treated by stem cell therapy. Preferably, the cell therapy composition of this invention is applied to the treatment of neuronal diseases, cardiac ischemic injury or cardiomyopathy, injury of spinal column and degenerative rhinitis.

The amount of dCNT in the cell therapy composition per ml of the aqueous ECM composition is in the range of 0.002-60 mg/ml, preferably, 0.01-10 mg/ml, more preferably, 0.01-1 mg/ml, and most preferably 0.01-0.3 mg/ml.

In the cell therapy compositions of this invention, a pharmaceutically acceptable carrier may be conventional for formulation, including carbohydrates (e.g., lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil, but not limited to the pharmaceutical compositions of this invention, further may contain wetting agent, lubricant, stabilizer, or mixtures of these substances. Details of suitable pharmaceutically acceptable carriers and formulations can be found in Remington's Pharmaceutical Sciences (19th ed., 1995), which is incorporated herein by reference.

The correct dosage of the pharmaceutical compositions of this invention will be varied according to the particular formulation, the mode of application, age, body weight, and gender of the patient, diet, time of administration, route of administration, condition of the patient, excretion rate, reaction sensitivity and so on.

According to conventional techniques known to those skilled in the art, the cell therapy compositions of this invention can be formulated with pharmaceutical acceptable carrier and/or vehicle, finally providing several forms including a unit dosage form or a multi-unit dosage forms. The dosage forms can comprise a solution, a suspension or an emulsion in an oily or aqueous medium as well as further dispersions or stabilizers.

The cell therapy composition of this invention promotes the formation of networking between stem cells transplanted and surrounding tissues, allowing stem cells transplanted to fully exert their functions and effects.

Other aspects of the invention involving discrete carbon nanotubes in an ECM application include a controlled drug delivery system containing dCNT and at least one active drug ingredient, preferably wherein the active drug ingredient is substantially associated with the DCNT that targets specific sites in the body (e.g., protein). The controlled drug delivery method can be activated by such methods as, but not limited to, heat, static or oscillating electromagnetic fields.

Another aspect of the ECM application is a bone adhesive composition containing dCNT and a biocompatible adhesive, for example, but not limited to, polyvinylalcohol (PVA), calcium phosphates, or polyurethanes, preferably activated by electro-magnetic radiation (e.g., x-rays, infra-red or ultraviolet radiation).

Structural bone systems and compositions containing dCNT, nerve repair containing dCNT and a biocompatible covering containing dCNT are also within the purview of the invention. These systems and compositions can be activated by radiation such as electro-magnetic radiation, x-rays, infra-red or ultraviolet radiation.

In addition to scaffolding, the invention also includes a stem cell growth and/or delivery substrate comprising dCNT.

The ECM containing dCNT can also comprise a film, cellular or fiber structure suitable for cell proliferation. A fibrous polymer may be included within the composition comprising discrete carbon nanotubes such as, but not limited to, electrospun PVA, silk or fibroin.

The film, fiber or foam ECM structures containing discrete carbon nanotubes may also comprise layers differing in composition. For example, in a tape the layers may consist of different amounts of discrete carbon nanotubes, different types of discrete carbon nanotubes, or different additives or concentration of additives.

A useful method for differentiating stem cells may include first putting the stem cells in or on a chosen ECM comprising discrete carbon nanotubes, culturing the stem cells to create the desired type of differentiated cell growth, then transplanting the differentiated cells into the living entity.

### EXAMPLES

The following example describes how to make a composition of this invention. The synthetic ECM material used is a commercial product known as Vitrogel, matrix material that primarily consists of crosslinked polysaccharides. The discrete carbon nanotubes can be made by means such as, but not limited to high pressure mixing or sonication. In this example discrete, oxidized multiwalled carbon nanotubes (dMWCNTs) of diameter about 13 nm were made using concentrated nitric acid at 95° C. for 2.5 hours followed by washing with de-ionized water to a pH 3.5. 2.3% by weight of oxidized species were determined by thermogravimetric analyses in nitrogen in the temperature range 200-600° C. . . . The oxidized carbon nanotubes were then made into flowable slurries in water and subjected to high pressure mixing to give discrete oxidized carbon nanotubes. A similar procedure can be used to make discrete oxidized single wall carbon nanotubes as the discrete oxidized multiwall carbon nanotubes. Other functionalization chemistries can be employed such as but not limited to nitrene insertion reactions. The discrete oxidized multiwall carbon nanotubes are dispersed in water using 1,2-distearoyl-sn-glycero-3-phosphoethanolamine-N-amino (polyethylene glycol) DSPE-PEG(NH₂) of 2 kDa molecular weight, in a dMWCNT:DSPE-PEG(NH₂) ratio of 1:0.55. The cells incorporated into the ECM were mesenchymal stem cells.

A Vitrogel solution (500 micrograms in 400000 micrograms of water was brought to room temperature. A cell suspension was prepared at 1×106 cells/ml. 150 μl of the cell suspension and Vitrogel solution was plated into each well of a 48 well cell culture plate, ensuring even coverage of the mixture at the bottom of the well. The gel formed after about 15 minutes. Without disturbing the gel an additional 150 μl of cell media with 0.002 mg/ml of discrete carbon nanotubes added to the top of the gel. The final concentration of dMWCNTs that are integrated into the ECM structure is 0.001 mg/ml of the gel, 0.06% by dry weight of the Vitrogel. The Vitrogel is 0.125% by weight relative to the total solution. The mass of discrete carbon nanotube, Vitrogel and water is provided in Table 1.

TABLE 1

| dMWCNT Mass (μg) | Synthetic Vitrogel Solids (μg) | Water (μg) |
|---|---|---|
| 0.3 | 500 | 400000 |

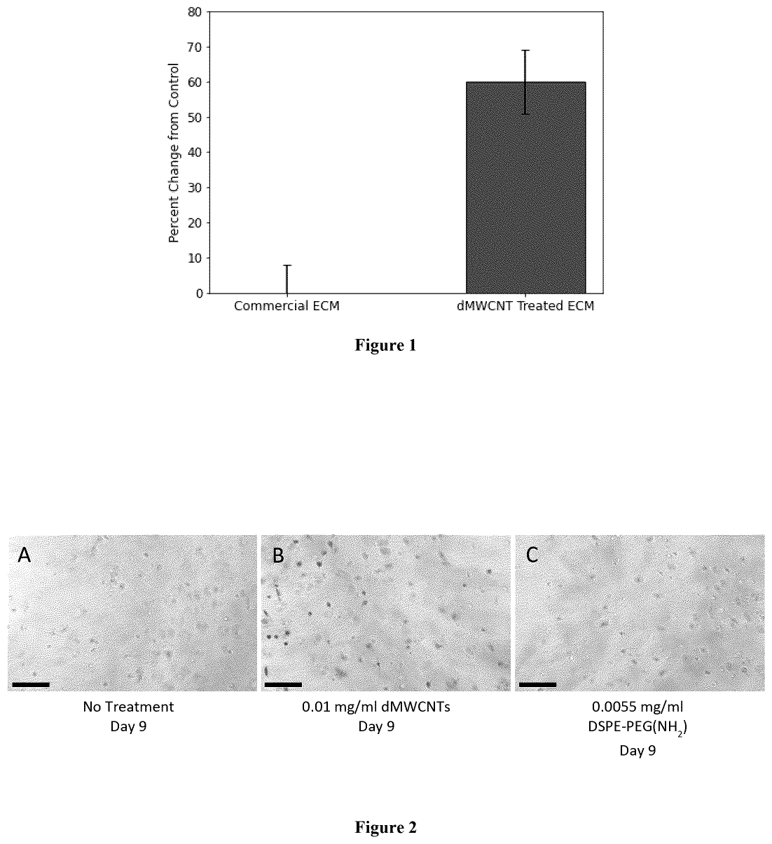

A control is made as Example 1 but without discrete multiwall carbon nanotubes being present. FIG. 1 shows Example 1 gives about a 60% improvement in Mesenchymal Stem Cell proliferation when incubated in a 3D ECM-like matrix containing discrete carbon nanotubes for 10 days compared to the control without discrete carbon nanotubes after being incubated for 10 days. The number of cells are measured using a colorimetric assay. The brightfield images of Mesenchymal Stem Cells incubated with and without discrete carbon nanotubes after 9 days are seen in FIG. 2.

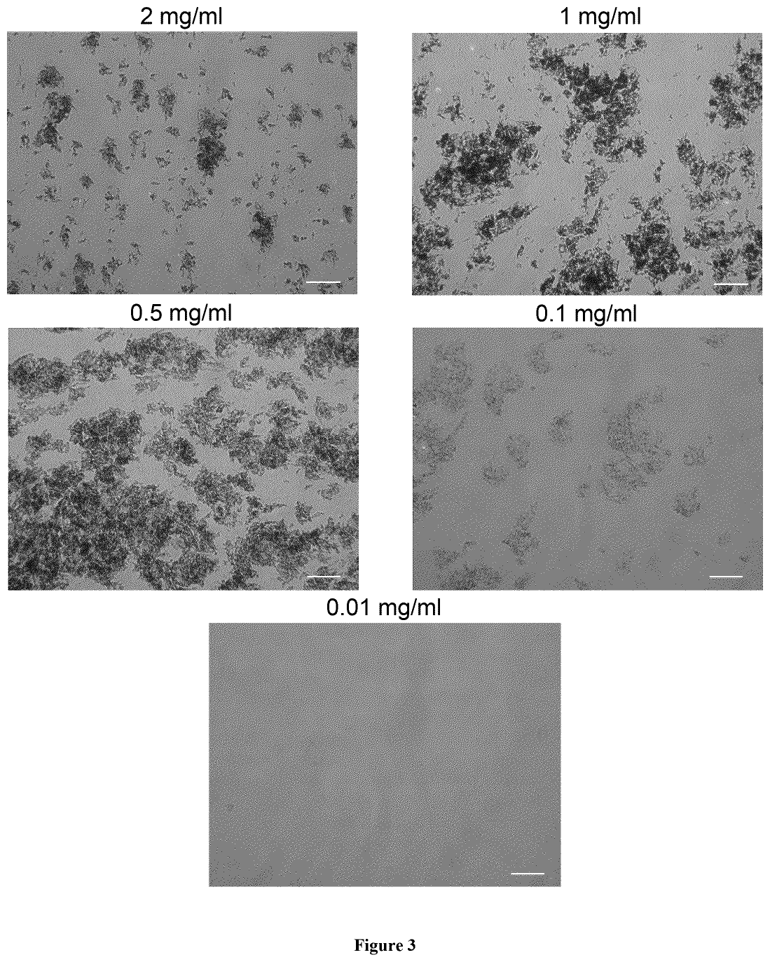

An example is made of an extracellular matrix comprising discrete carbon nanotubes in which there are no living cells present and the nanotubes only remain discrete at certain concentrations. In a method similar to that outlined in Table 1, a Vitrogel matrix was prepared and combined with discrete multiwalled carbon nanotubes dispersed in DSPE-PEG(NH₂) (molecular weight 2 kDa) and branched polyethylenimine (PEI, molecular weight 270 kDa), in a dMWCNT:DSPE-PEG(NH₂):PEI ratio of 10:7:3 across a range of concentrations. The concentration of dMWCNTs was varied from 0.01, 0.1, 0.5, 1, and 2 mg/ml, while the concentration of Vitrogel components was kept constant. The mixture was incubated at 37° C. for 24 hours. FIG. 3 shows brightfield microscopy images of each concentration of dMWCNTs in Vitrogel. The scale bar is 100 μm. For all concentrations except 0.01 mg/ml, significant aggregation is visible, indicating that the MWCNTs used under these conditions are not well dispersed. However, there is no visible aggregation at the 0.01 mg/ml concentration, thus providing an example of the well dispersed dMWCNTs incorporated into an ECM described by this patent.

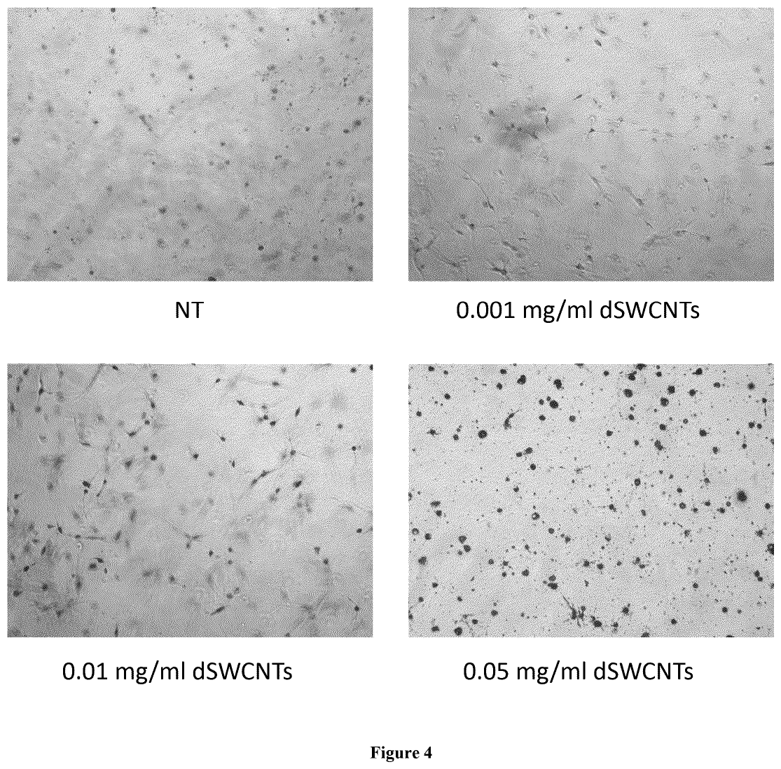

A further example is shown in FIG. 4 wherein single walled carbon nanotubes noncovalently dispersed in DSPE-PEG(NH₂). (SWCNT:DSPE-PEG mass ratio 1:0.55) were integrated into Vitrogel synthetic ECM at three concentrations (0.001, 0.01, and 0.05 mg/ml) as previously described. MSCs were seeded into the Vitrogel, incubated for 3 days at 37° C., and imaged under brightfield microscopy. At concentrations of 0.001-0.01 mg/ml, the number of visible cells was higher than those in the no treatment group. Furthermore, the amount of elongation, a measure of MSC viability, of cells in the 0.001-0.01 mg/ml range was visibly higher than the no treatment group, suggesting a positive effect on MSC proliferation due to the presence of dSWCNTs.

### EMBODIMENTS

One embodiment of this invention is a composition comprising a mixture of an extracellular matrix and discrete carbon nanotubes wherein the extracellular matrix comprises components wherein the components are selected from the class of proteins, proteoglycans polysaccharides, lipids, peptides, messenger molecules, signaling molecules, or any mixture thereof, and wherein the discrete carbon nanotubes are less than about 1% by weight of the dry weight of the total composition.

Another embodiment of this invention is a composition comprising a mixture of an extracellular matrix and discrete carbon nanotubes wherein at least a portion of the extracellular matrix, or at least a portion of the discrete carbon nanotubes, or both, are at least partially oriented.

Yet another embodiment of this invention is a composition comprising a mixture of an extracellular matrix and discrete carbon nanotubes wherein at least a portion of the discrete carbon nanotubes are physically adhered directly, or indirectly, to at least one component of the composition, or optionally at least a portion of the discrete carbon nanotubes are chemically bonded to at least one component of the composition.

Another embodiment of this invention is a composition comprising a mixture of an extracellular matrix and discrete carbon nanotubes further comprising living cells. The living cells can be stem cells, preferably primary stem cells.

An embodiment of this invention is a composition comprising a mixture of an extracellular matrix and discrete carbon nanotubes for use in injury, burns, and wound care, sensors, and bone, skin, muscle, nerve, blood, and organ treatment for accelerated repair of damage compared to a similar composition of the extracellular matrix without discrete carbon nanotubes.

An additional embodiment of this invention is that the composition can be shaped, injected, implanted or fitted. The form of the composition can be a closed cell foam, an open cell foam, a film, a strand, particles, or mixtures thereof.

Another embodiment of this invention is a composition comprising a mixture of an extracellular matrix and discrete carbon nanotubes wherein the composition is subjected to a static current, an oscillating current, a static voltage, an oscillating voltage, or any combination thereof. The static or oscillating voltage supplied is preferably less than about 100 millivolts, more preferably less than about 50 millivolts, and most preferably less than about 10 millivolts.

Another embodiment of this invention is a composition with stems cells wherein the stem cells grow at least about 10% faster, preferably at least about 20% faster and more preferably at least about 50% faster than in a comparable composition without the discrete carbon nanotubes in substantially the same media under substantially the same conditions.

An embodiment of this invention is a composition comprising a mixture of an extracellular matrix and discrete carbon nanotubes wherein the discrete carbon nanotubes are substantially individualized from other discrete carbon nanotubes in the extracellular matrix.

A further embodiment of this invention is a composition comprising a mixture of an extracellular matrix and discrete carbon nanotubes wherein the composition further comprises a dispersing agent wherein the dispersing agent substantially prevents the discrete carbon nanotubes from agglomerating. The dispersing agent is selected from the class of anionic, cationic, non-ionic and zwitterionic molecules.

An additional embodiment of this invention is a composition comprising a mixture of an extracellular matrix and discrete carbon nanotubes wherein at least a portion of the discrete carbon nanotubes further comprise a molecule distinct in structure from the components of the extracellular matrix wherein the molecule is at least partially bound to the nanotube surface and is selected from the class of molecules that function as an antibacterial, a growth factor, a growth stimulant, a nutrient, or combinations thereof.

Another embodiment of this invention is a composition comprising a mixture of an extracellular matrix and discrete carbon nanotubes wherein the composition further comprises graphene, graphene oxide, carbon nanofibers, cellulose nanofibers, hydroxy apatite, silk, and mixtures thereof.

Yet another embodiment of this invention is a composition comprising mixture of an extracellular matrix and discrete carbon nanotubes further comprising water.

Further embodiments relate to the method for growing living cells comprising a mixture of an extracellular matrix and discrete carbon nanotubes and living cells wherein the extracellular matrix comprises components wherein the components are selected from proteins, proteoglycans polysaccharides, lipids, peptides, messenger molecules, signaling molecules, or any mixture thereof, and wherein the discrete carbon nanotubes are less than about 1% by dry weight of the total composition, wherein the discrete carbon nanotubes are less than about 1% by dry weight of the total composition; and wherein the amount of living cells is sufficient to allow growth proliferation; and providing conditions sufficient to facilitate growth of living cells and growing additional living cells. At least a portion of the discrete carbon nanotubes may coat the extracellular matrix or be present as a more concentrated layer.

A further embodiment of this invention is a method of harvesting at least a portion of the grown living cells or harvesting of one or more biological products produced by the living cells. Optionally the living cells are stem cells and preferably are primary stem cells. The stems cells may be differentiated or not.

Another embodiment of this invention is a method comprising injecting at least a portion of the grown living cells into an animal.

Yet another embodiment of this invention comprising a mixture of an extracellular matrix and discrete carbon nanotubes is a method to add a molecule distinct in structure from the components of the extracellular matrix wherein the molecule is selected from the class of molecules that function as an antibacterial, a growth factor, a growth stimulant, a nutrient, or combinations thereof. Furthermore, a dispersing agent may be optionally added to the discrete carbon nanotubes prior to, at the same time as, or after adding the molecule distinct in structure from the components of the extracellular matrix.

### SPECIFIC EMBODIMENTS

1. A composition comprising:
a mixture of an extracellular matrix and discrete carbon nanotubes wherein the extracellular matrix comprises components selected from the class of proteins, proteoglycans polysaccharides, lipids, peptides, messenger molecules, signaling molecules, or any mixture thereof, and wherein the discrete carbon nanotubes are less than about 1% by weight of the dry weight of the total composition.

2. The composition of embodiment 1 wherein at least a portion of the extracellular matrix, or at least a portion of the discrete carbon nanotubes, or both, are at least partially oriented.

3. The composition of embodiment 1 wherein at least a portion of the discrete carbon nanotubes are physically adhered directly, or indirectly, to at least one component of the composition.

4. The composition of embodiment 1 wherein at least a portion of the discrete carbon nanotubes are chemically attached to a component of the composition.

5. The composition of embodiment 1 further comprising living cells.

6. The composition of embodiment 5 further comprising stem cells.

7. The composition of embodiment 1 for use in injury, burns, or wound care.

8. The composition of embodiment 1 for use in sensors.

9. The composition of embodiment 1 for use in bone, skin, muscle, nerve, blood, and organ treatment for accelerated repair of damage compared to a similar composition of the extracellular matrix without discrete carbon nanotubes.

10. The composition of embodiment 1 wherein the composition can be shaped, injected, implanted or fitted.

11. The composition of embodiment 1 wherein the composition is subjected to a static current, an oscillating current, a static voltage, an oscillating voltage, or any combination thereof.

12. The composition of embodiment 6 wherein the stem cells are primary stem cells.

13. The composition of embodiment 6 wherein the stems cells grow at least 10% faster than in a comparable composition without the discrete carbon nanotubes in substantially the same media under substantially the same conditions.

14. The composition of embodiment 6 wherein the stems cells grow at least 50% faster than in a comparable composition without the discrete carbon nanotubes in substantially the same media under substantially the same conditions.

15. The composition of embodiment 1 wherein a majority, or at least 60%, or at least 70%, or at least 80% or more of the discrete carbon nanotubes are substantially individualized from other discrete carbon nanotubes in the extracellular matrix and/or remain substantially individualized from other discrete carbon nanotubes in the extracellular matrix.

16. The composition of embodiment 1 wherein the composition further comprises one or more dispersing agents wherein the dispersing agents substantially prevent the discrete carbon nanotubes from agglomerating.

17. The composition of embodiment 16 wherein the one or more dispersing agents are selected from the class of anionic, cationic, non-ionic, amphiphilic and zwitterionic molecules.

18. The composition of embodiment 1 wherein at least a portion of the discrete carbon nanotubes further comprise a molecule distinct in structure from the components of the extracellular matrix wherein the molecule is at least partially bound to the nanotube surface and is selected from the class of molecules that function as an antibacterial, a growth factor, a growth stimulant, a nutrient, or combinations thereof.

19. The composition of embodiment 1 wherein the composition further comprises graphene, graphene oxide, carbon nanofibers, cellulose nanofibers, hydroxy apatite, silk, and mixtures thereof.

20. The composition of embodiment 1 in the form of a closed cell foam, an open cell foam, a film, a strand, particles, or mixtures thereof.

21. The composition of embodiment 1 further comprising water.

22. A method for growing living cells comprising:
a mixture of an extracellular matrix and discrete carbon nanotubes and living cells wherein the extracellular matrix comprises components selected from the class of proteins, proteoglycans polysaccharides, lipids, peptides, messenger molecules, signaling molecules, or any mixture thereof, and wherein the discrete carbon nanotubes are less than about 1% by dry weight of the total composition, wherein the discrete carbon nanotubes are less than about 1% by dry weight of the total composition; and wherein the amount of living cells is sufficient to allow growth proliferation; and providing conditions sufficient to facilitate growth of living cells and growing additional living cells.

23. The method of embodiment 22 wherein at least a portion of the discrete carbon nanotubes coat the extracellular matrix or form a layer.

24. The method of embodiment 23 further comprising harvesting at least a portion of the grown living cells.

25. The method of embodiment 24 further comprising the harvesting of one or more biological products produced by the living cells.

26. The method of embodiment 25 wherein the living cells are stem cells.

27. The method of embodiment 25 wherein the living cells are primary stem cells

28. The method of embodiment 26 wherein the stem cells are differentiated.

29. The method of embodiment 26 wherein the stem cells are not differentiated.

30. The method of embodiment 26 further comprising injecting at least a portion of the grown stem cells into an animal.

31. The method of embodiment 22 further comprising a dispersing agent and a molecule distinct in structure from the components of the extracellular matrix wherein the molecule is selected from the class of molecules that function as an antibacterial, a growth factor, a growth stimulant, a nutrient, or combinations thereof.

32. The method 31 wherein the dispersing agent is optionally added to the discrete carbon nanotubes prior to, at the same time as, or after adding the molecule distinct in structure from the components of the extracellular matrix.

33. The composition of embodiment 1 wherein the discrete carbon nanotubes are selected from the class of single walled, double walled, multiwalled carbon nanotubes or any combination thereof.

34. The composition of embodiment 1 wherein the discrete carbon nanotubes are functionalized in the range of 1 μmol per gram of dry nanotubes to 1 mmol per gram of dry nanotubes by functional groups selected from the class of carboxyls, hydroxyls, esters, ethers, imines, amines, amides, phosphates, sulfates, nitrates, or combinations thereof.

35. The composition of embodiment 5 wherein at least a portion the living cells maintain viability at least for an additional 2 weeks, or at least an additional 3 weeks, or at least an additional 4 weeks or more after incorporation into the extracellular matrix compared to an extracellular matrix of similar composition without discrete carbon nanotubes.

What is claimed is:

1. A composition comprising:
an extracellular matrix; and
discrete carbon nanotubes;
wherein the extracellular matrix comprises one or more components selected from the classes of proteins, proteoglycans polysaccharides, lipids, peptides, messenger molecules, signaling molecules, or any mixture thereof;
wherein the discrete carbon nanotubes are less than about 1% by weight of the dry weight of the total composition.

2. The composition of claim 1 wherein at least a portion of the extracellular matrix, or at least a portion of the discrete carbon nanotubes, or both, are at least partially oriented.

3. The composition of claim 1 wherein at least a portion of the discrete carbon nanotubes are physically adhered directly, or indirectly, to at least one component of the composition.

4. The composition of claim 1 wherein at least a portion of the discrete carbon nanotubes are chemically attached to a component of the composition.

5. The composition of claim 1 further comprising living cells.

6. The composition of claim 5 wherein at least a portion of the living cells comprise stem cells.

7. The composition of claim 1 wherein the composition is in a shaped form, an injectable form, an implantable form, or a fitted form.

8. The composition of claim 6 wherein the stem cells are primary stem cells.

9. The composition of claim 6 wherein the stems cells grow at least 10% faster than in a comparable composition without the discrete carbon nanotubes in substantially the same media under substantially the same conditions.

10. The composition of claim 5 wherein there is a sufficient amount of the discrete carbon nanotubes that are substantially individualized from other discrete carbon nanotubes in the extracellular matrix such that the living cells grow at least 10% faster than in a comparable composition without the discrete carbon nanotubes in substantially the same media under substantially the same conditions.

11. The composition of claim 1 wherein the composition further comprises one or more dispersing agents wherein the one or more dispersing agents substantially prevent the discrete carbon nanotubes from agglomerating.

12. The composition of claim 11 wherein the one or more dispersing agents are selected from the class of anionic, cationic, non-ionic, amphiphilic and zwitterionic molecules.

13. The composition of claim 1 wherein at least a portion of the discrete carbon nanotubes further comprise a molecule distinct in structure from the components of the extracellular matrix wherein the molecule is at least partially bound to a surface of the discrete carbon nanotube surface and is selected from the class of molecules that function as an antibacterial, a growth factor, a growth stimulant, a nutrient, or combinations thereof.

14. The composition of claim 1 wherein the composition further comprises graphene, graphene oxide, carbon nanofibers, cellulose nanofibers, hydroxy apatite, silk, and mixtures thereof.

15. The composition of claim 1 wherein the discrete carbon nanotubes are single walled carbon nanotubes, double walled carbon nanotubes, few walled carbon nanotubes, multiwalled carbon nanotubes, or any combination thereof.

16. The composition of claim 1 wherein at least a portion of the discrete carbon nanotubes are functionalized by one or more functional groups.

17. The composition of claim 16 wherein the one or more functional groups are selected from the class of carboxyls, hydroxyls, esters, ethers, imines, amines, amides, phosphates, sulfates, nitrates, or any combination thereof

18. The composition of claim 16 wherein the portion of the functionalized discrete carbon nanotubes are functionalized in the range of 1 μmol per gram of dry nanotubes to 1 mmol per gram of dry nanotubes.

19. The composition of claim 17 wherein the portion of the functionalized discrete carbon nanotubes are functionalized in the range of 1 μmol per gram of dry nanotubes to 1 mmol per gram of dry nanotubes.

20. The composition of claim 5 wherein at least a portion the living cells maintain viability at least for an additional 2 weeks after incorporation into the extracellular matrix compared to an extracellular matrix of similar composition without discrete carbon nanotubes.
